(19) United States
(12) Patent Application Publication
(10) Pub. No.: US 2024/0408348 A1
(43) Pub. Date: Dec. 12, 2024

(54) Catheters, Catheter Assemblies, and Methods for Mechanically Inhibiting Formation of Occlusions

(71) Applicant: Bard Access Systems, Inc., Salt Lake City, UT (US)

(72) Inventors: Syed F. Ahmed, Bangalore (IN); Sharath Umesh, Bangalore (IN); Shishir Prasad, Ramsey, NJ (US)

(21) Appl. No.: 18/736,357

(22) Filed: Jun. 6, 2024

Related U.S. Application Data
(60) Provisional application No. 63/471,744, filed on Jun. 7, 2023.

(51) Int. Cl. A61M 25/00

(52) U.S. Cl. CPC A61M 25/0021; A61M 25/0009; A61M 25/0097; A61M 2205/0294; A61M 2205/8206

(57) ABSTRACT

A catheter assembly can include a catheter, a controller, and an internal power source. The catheter can include a catheter tube and a catheter hub, wherein a proximal-end portion of the catheter tube can be disposed in the catheter hub. The catheter tube can incorporate a plurality of piezoelectric transducers into a length of the catheter tube. The plurality of piezoelectric transducers can be configured as a plurality of vibrators for vibrating and, thereby, inhibiting buildup of biomaterial on a luminal or abluminal surface of the catheter tube by way of vibrations along the length of the catheter tube when the catheter tube is placed in a vasculature. The controller can include a processor and memory, wherein the controller can be configured to control at least the plurality of piezoelectric transducers. The internal power source can be configured to power the controller and the plurality of piezoelectric transducers.

### PRIORITY

This application claims the benefit of priority to U.S. Provisional Application No. 63/471,744, filed Jun. 7, 2023, which is incorporated by reference in its entirety into this application.

### BACKGROUND

Occlusions in catheters including central venous catheters (“CVCs”) and peripherally inserted central catheters (“PICCs”) can inhibit or even prevent fluids from flowing through such catheters. This can lead to ineffective medication therapy as well as complications such as infiltration, phlebitis, or infection. The foregoing occlusions can arise in a variety of different ways. In an example, thrombotic occlusions can arise when one or more thrombi form within, around, or at a distal-end portion of a catheter as shown in FIG. 8. In another example, non-thrombotic occlusions can arise from biofilm formation, lipid residue from lipid-containing nutritional admixtures or drug formulations including oleaginous vehicles, or drug precipitation from the same or different drug formulations within, around, or at a distal-end portion of a catheter. What is needed are catheters, catheter assemblies, and methods for inhibiting formation of such occlusions. Disclosed herein are catheters, catheter assemblies, and methods that address the foregoing by mechanically inhibiting the formation of such occlusions.

### SUMMARY

Disclosed herein is a catheter assembly including, in some embodiments, a catheter, a controller, and an internal power source. The catheter includes a catheter tube and a catheter hub, a proximal-end portion of the catheter tube disposed in the catheter hub. The catheter tube incorporates a plurality of piezoelectric transducers into a length of the catheter tube. The plurality of piezoelectric transducers is configured as a plurality of vibrators for vibrating and, thereby, inhibiting buildup of biomaterial on a luminal surface of the catheter tube, an abluminal surface of the catheter tube, or both the luminal and abluminal surfaces of the catheter tube by way of vibrations along the length of the catheter tube when the catheter tube is placed in a vasculature. The controller includes a processor and memory, the controller configured to control at least the plurality of piezoelectric transducers. The internal power source is configured to power the controller and the plurality of piezoelectric transducers.

In some embodiments, the plurality of piezoelectric transducers and electrical leads connecting the plurality of piezoelectric transducers to the controller are flexible electronics. The flexible electronics are fabricated on the luminal surface of the catheter tube, fabricated on the abluminal surface of the catheter tube, or disposed between the luminal and abluminal surfaces of the catheter tube.

In some embodiments, the internal power source is a lithium-ion battery or a flexible battery.

In some embodiments, a thickness of the flexible electronics ranges from about 1 μm to about 500 μm.

In some embodiments, individual piezoelectric transducers of the plurality of piezoelectric transducers, individual groups of piezoelectric transducers of the plurality of piezoelectric transducers, or a combination of the individual piezoelectric transducers and the individual groups of piezoelectric transducers are individually electronically addressed.

In some embodiments, the individual piezoelectric transducers, the individual groups of piezoelectric transducers, or the combination of the individual piezoelectric transducers and the individual groups of piezoelectric transducers being individually electronically addressed allows the vibrations along the length of the catheter tube to be customized by location of the individual piezoelectric transducers or the individual groups of piezoelectric transducers.

In some embodiments, the individual piezoelectric transducers, the individual groups of piezoelectric transducers, or the combination of the individual piezoelectric transducers and the individual groups of piezoelectric transducers being individually electronically addressed allows a distal-end portion of the catheter tube to be excised for a desired placement length of the catheter tube. Remaining piezoelectric transducers of the plurality of piezoelectric transducers not excised with the distal-end portion of the catheter tube are capable of vibrations along the placement length of the catheter tube in accordance with being individually electronically addressed.

In some embodiments, the plurality of piezoelectric transducers are further configured as a plurality of sensors for sensing and, thereby, monitoring environmental conditions of the catheter tube when the catheter tube is placed in the vasculature.

In some embodiments, the controller and the internal power source are incorporated into a portion of the catheter hub. Optionally, the controller and the internal power source are incorporated into a suture wing of the catheter hub.

In some embodiments, the controller and the internal power source are incorporated into a base plate or top cover of a catheter securement device. The base plate or top cover of the catheter securement device include securement device-based electrical connectors for connecting to catheter-based electrical connectors for powering and controlling the plurality of piezoelectric transducers.

In some embodiments, the controller is configured to vibrate the plurality of piezoelectric transducers in a frequency range from about 30 Hz to about 15 MHz with an amplitude range from about 1 nm to about 100 μm.

In some embodiments, the buildup of biomaterial is an intraluminal thrombus, a fibrin tail, a fibrin sheath, a mural thrombus, a biofilm, or a combination thereof.

In some embodiments, the catheter is a CVC or a PICC.

Also disclosed herein is a method of catheter assembly including, in some embodiments, a placing operation and a powering operation. The placing operation includes placing a catheter of the catheter assembly in a vasculature of a patient. The catheter includes a catheter tube and a catheter hub, a proximal-end portion of the catheter tube disposed in the catheter hub. The catheter tube incorporates a plurality of piezoelectric transducers into a length of the catheter tube. The plurality of piezoelectric transducers is configured as a plurality of vibrators. The powering operation includes powering up the catheter assembly by way of an internal power source. The powering operation activates a controller including a processor and memory configured to control the plurality of piezoelectric transducers, thereby vibrating and, thusly, inhibiting buildup of biomaterial on a luminal surface of the catheter tube, an abluminal surface of the catheter tube, or both the luminal and abluminal surfaces of the catheter tube by way of vibrations along the length of the catheter tube.

In some embodiments, the plurality of piezoelectric transducers and electrical leads connecting the plurality of piezoelectric transducers to the controller are flexible electronics. The flexible electronics are fabricated on the luminal surface of the catheter tube, fabricated on the abluminal surface of the catheter tube, or disposed between the luminal and abluminal surfaces of the catheter tube.

In some embodiments, individual piezoelectric transducers of the plurality of piezoelectric transducers, individual groups of piezoelectric transducers of the plurality of piezoelectric transducers, or a combination of the individual piezoelectric transducers and the individual groups of piezoelectric transducers are individually electronically addressed.

In some embodiments, the individual piezoelectric transducers, the individual groups of piezoelectric transducers, or the combination of the individual piezoelectric transducers and the individual groups of piezoelectric transducers being individually electronically addressed allows the vibrations along the length of the catheter tube to be customized by location of the individual piezoelectric transducers or the individual groups of piezoelectric transducers.

In some embodiments, the method further comprises an excising operation before the placing operation of placing the catheter in the vasculature of a patient. The excising operation includes excising a distal-end portion of the catheter tube for a desired placement length of the catheter tube. The individual piezoelectric transducers, the individual groups of piezoelectric transducers, or the combination of the individual piezoelectric transducers and the individual groups of piezoelectric transducers being individually electronically addressed allows remaining piezoelectric transducers of the plurality of piezoelectric transducers not excised with the distal-end portion of the catheter tube to vibrate along the placement length of the catheter tube after the powering operation of powering up the catheter assembly in accordance with being individually electronically addressed.

In some embodiments, the plurality of piezoelectric transducers are further configured as a plurality of sensors for sensing and, thereby, monitoring environmental conditions of the catheter tube upon the placing operation of placing the catheter in the vasculature of the patient and the powering operation of powering up the catheter assembly and the activating of the controller.

In some embodiments, the controller and the internal power source are incorporated into a portion of the catheter hub. Optionally, the controller and the internal power source are incorporated into a suture wing of the catheter hub.

In some embodiments, the buildup of biomaterial is an intraluminal thrombus, a fibrin tail, a fibrin sheath, a mural thrombus, a biofilm, or a combination thereof.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a catheter for mechanically inhibiting formation of occlusions in accordance with some embodiments.

FIG. 2 illustrates a detailed view of a catheter tube of the catheter including a plurality of piezoelectric transducers along a length of the catheter tube in accordance with some embodiments.

FIG. 3 illustrates a detailed view of a catheter hub of the catheter including a controller and an internal power source incorporated into a portion of the catheter hub in accordance with some embodiments.

FIG. 4 illustrates another catheter for mechanically inhibiting formation of occlusions in accordance with some embodiments.

FIG. 5 illustrates a detailed view of a base plate of a catheter securement device for the catheter of FIG. 4, the catheter securement device including the controller and the internal power source incorporated into the base plate in accordance with some embodiments.

FIG. 6 illustrates the catheter of FIG. 4 secured in the catheter securement device of FIG. 5, thereby forming a catheter assembly for mechanically inhibiting formation of occlusions in accordance with some embodiments.

FIG. 7 illustrates a block diagram of the catheter of FIG. 1 or the catheter assembly of FIG. 6 in accordance with some embodiments.

FIG. 8 illustrates various thrombotic occlusions in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal-end portion,” a “proximal-end section,” or a “proximal-end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal-end portion,” a “distal-end section,” or a “distal-end length” of the medical device, respectively.

Unless defined otherwise, all technical and scientific terms used herein have the same meaning as commonly understood by those of ordinary skill in the art.

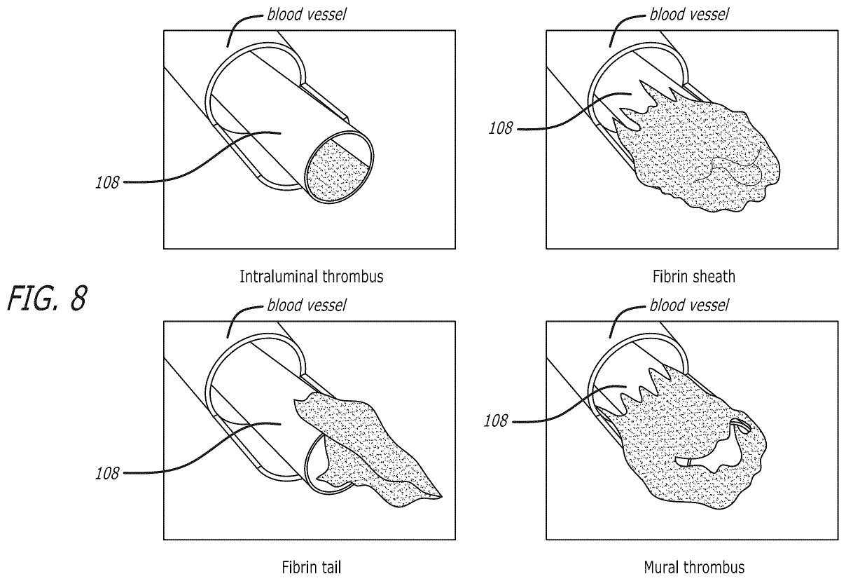

Occlusions in catheters including CVCs and PICCs can inhibit or even prevent fluids from flowing through such catheters. This can lead to ineffective medication therapy as well as complications such as infiltration, phlebitis, or infection. The foregoing occlusions can arise in a variety of different ways. In an example, thrombotic occlusions can arise when one or more thrombi form within, around, or at a distal-end portion of a catheter as shown in FIG. 8. In another example, non-thrombotic occlusions can arise from biofilm formation, lipid residue from lipid-containing nutritional admixtures or drug formulations including oleaginous vehicles, or drug precipitation from the same or different drug formulations within, around, or at a distal-end portion of a catheter. What is needed are catheters, catheter assemblies, and methods for inhibiting formation of such occlusions. Disclosed herein are catheters, catheter assemblies, and methods that address the foregoing by mechanically inhibiting the formation of such occlusions.

For example, a catheter assembly can include a catheter, a controller, and an internal power source. The catheter can include a catheter tube and a catheter hub, wherein a proximal-end portion of the catheter tube can be disposed in the catheter hub. The catheter tube can incorporate a plurality of piezoelectric transducers into a length of the catheter tube. The plurality of piezoelectric transducers can be configured as a plurality of vibrators for vibrating and, thereby, inhibiting buildup of biomaterial on a luminal or abluminal surface of the catheter tube by way of vibrations along the length of the catheter tube when the catheter tube is placed in a vasculature. The controller can include a processor and memory, wherein the controller can be configured to control at least the plurality of piezoelectric transducers. The internal power source can be configured to power the controller and the plurality of piezoelectric transducers. Such a catheter assembly and features thereof will become more apparent to those of skill in the art in view of the accompanying drawings and following description.

### Catheters

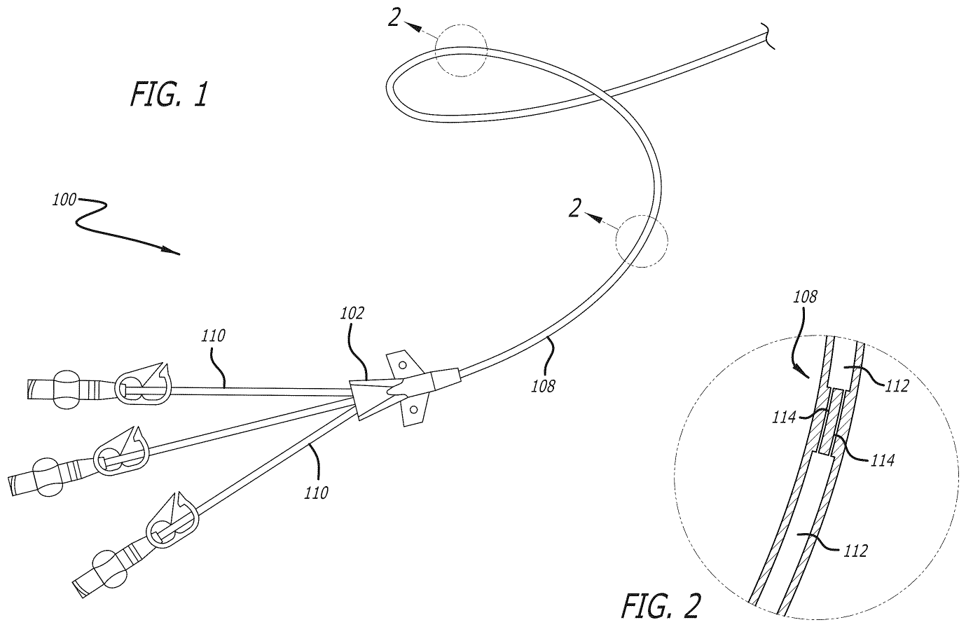

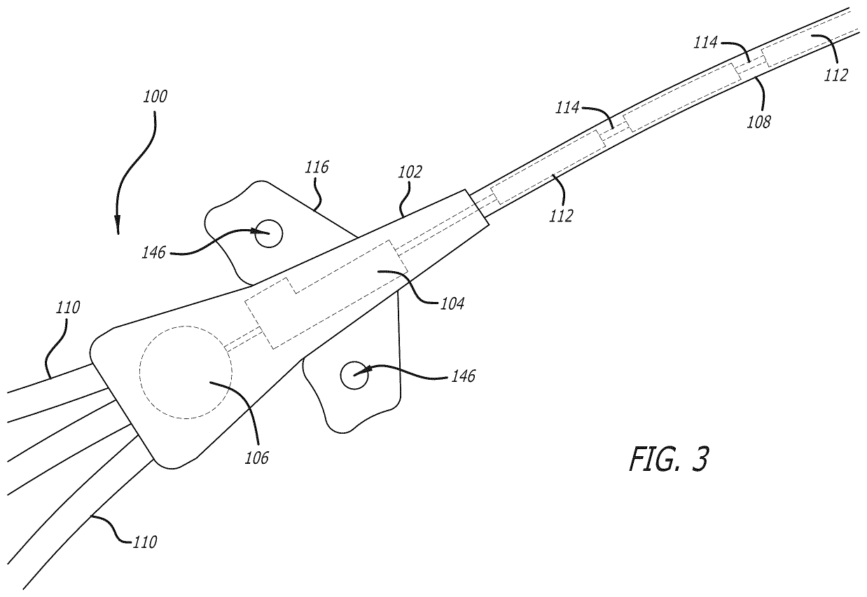

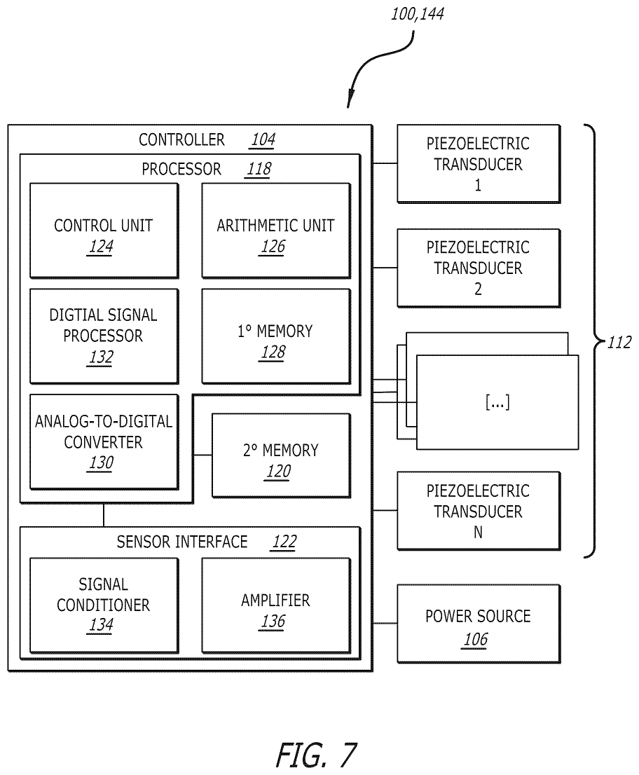

FIG. 1 illustrates a catheter 100 for mechanically inhibiting formation of occlusions in accordance with some embodiments. FIG. 3 illustrates a detailed view of a catheter hub 102 of the catheter 100 including a controller 104 and an internal power source 106 incorporated into a portion of the catheter hub 102 in accordance with some embodiments. FIG. 7 illustrates a block diagram of the catheter 100 of FIG. 1 in accordance with some embodiments.

As shown in FIGS. 1 and 3, the catheter 100 can include a catheter tube 108, the catheter hub 102, and one or more extension legs 110 coupled together in the foregoing order. Indeed, the catheter tube 108 can include a proximal-end portion disposed in the catheter hub 102, and each extension leg of the one-or-more extension legs 110 can include a distal-end portion disposed in the catheter hub 102. Such a catheter 100 can be a CVC or a PICC in some embodiments; however, it should be understood the catheter 100 is not limited thereto, as the catheter 100 can be any catheter intended for placement in a vasculature of a patient for a time sufficient for formation of thrombotic or non-thrombotic occlusions.

FIG. 2 illustrates a detailed view of the catheter tube 108 of the catheter 100 including a plurality of piezoelectric transducers 112 along a length of the catheter tube 108 in accordance with some embodiments.

As shown in FIG. 2, the catheter tube 108 can incorporate the plurality of piezoelectric transducers 112 into the foregoing length of the catheter tube 108. By way of the converse piezoelectric effect, the plurality of piezoelectric transducers 112 can be configured as a plurality of vibrators for vibrating and, thereby, inhibiting buildup of thrombotic or non-thrombotic occlusions on a luminal surface of the catheter tube 108, an abluminal surface of the catheter tube 108, or both the luminal and abluminal surfaces of the catheter tube 108 by way of adhesion-preventing vibrations along the length of the catheter tube 108 including the plurality of piezoelectric transducers 112 such as when the catheter tube 108 is placed in a vasculature of a patient for a period of time. For example, the plurality of piezoelectric transducers 112 configured as the plurality of vibrators can vibrate and, thereby, inhibit buildup of thrombotic biomaterial such as an intraluminal thrombus, a fibrin tail, a fibrin sheath, or a mural thrombus, each of which is shown in FIG. 8; non-thrombotic biomaterial such as a biofilms; or a combination thereof by way of the adhesion-preventing vibrations along the length of the catheter tube 108 including the plurality of piezoelectric transducers 112 when the catheter tube 108 is placed in a vasculature of a patient for a period of time. Being that the adhesion-preventing vibrations along the length of the catheter tube 108 inhibit buildup of non-thrombotic biomaterial such as biofilms, the catheter tube 108 need not be formed of an antimicrobial material, formed of an antimicrobial-containing material, coated with an antimicrobial coating, or any combination thereof.

The plurality of piezoelectric transducers 112 and any electrical leads 114 connecting the plurality of piezoelectric transducers 112 to the controller 104 can be implemented in flexible electronics, wherein the plurality of piezoelectric transducers 112 can be formed of lead zirconate titanate (“PZT”) or polyvinylidene fluoride (“PVDF”), and wherein each piezoelectric transducer of the plurality of piezoelectric transducers 112 can have same or different dimensions as another piezoelectric transducer of the plurality of piezoelectric transducers 112. Such flexible electronics can be fabricated on the luminal surface of the catheter tube 108, fabricated on the abluminal surface of the catheter tube 108, or disposed between the luminal and abluminal surfaces of the catheter tube 108. When the flexible electronics are fabricated on the luminal surface of the catheter tube 108, the catheter tube 108 can be extruded over a tubular polymeric substrate including the flexible electronics, the catheter tube 108 extruded thereover being of a same or different polymer as the tubular substrate. Alternatively, the tubular substrate including the flexible electronics can be dip coated in a coating mixture, again, of a same or different polymer as the tubular substrate. When the flexible electronics are disposed between the luminal and abluminal surfaces of the catheter tube 108, the flexible electronics can be fabricated on an inner layer of the catheter tube 108 with an outer layer of the catheter tube 108 extruded or dip coated thereover, the outer layer of catheter tube 108 being a same or different polymer as the inner layer of the catheter tube 108.

Notably, a thickness of the flexible electronics can be at least about 1 μm, including at least about 20 μm, such as at least about 50 μm, for example, at least about 80 μm, 100 μm, or 500 μm. Alternatively, the thickness of the flexible electronics can be no more than about 500 μm, including no more than about 100 μm, such as no more than about 80 μm, for example, no more than about 50 μm, 20 μm, or 1 μm. For example, the thickness of the flexible electronics can range from about 1 μm to about 500 μm.

Advantageously, individual piezoelectric transducers of the plurality of piezoelectric transducers 112, individual groups of piezoelectric transducers of the plurality of piezoelectric transducers 112, or a combination of the individual piezoelectric transducers and the individual groups of piezoelectric transducers can be configured to be individually electronically addressed by way of dedicated electrical leads 114 to each piezoelectric transducer of the plurality of piezoelectric transducers 112 or group of piezoelectric transducers of the plurality of piezoelectric transducers 112. The individual piezoelectric transducers, the individual groups of piezoelectric transducers, or the combination of the individual piezoelectric transducers and the individual groups of piezoelectric transducers being individually electronically addressable allows the adhesion-preventing vibrations along the length of the catheter tube 108 to be customized with respect to frequency, amplitude, or pulse waveform by location or specific sites of the individual piezoelectric transducers or the individual groups of piezoelectric transducers. Further, the individual piezoelectric transducers, the individual groups of piezoelectric transducers, or the combination of the individual piezoelectric transducers and the individual groups of piezoelectric transducers being individually electronically addressable allows a distal-end portion of the catheter tube 108 to be excised for a desired placement length of the catheter tube 108. Remaining piezoelectric transducers of the plurality of piezoelectric transducers 112 not excised with the distal-end portion of the catheter tube 108 remain capable of the adhesion-preventing vibrations along the placement length of the catheter tube 108 in accordance with being individually electronically addressable. As such, when the catheter 100 is configured as a PICC, the catheter 100 retains both the cut-to-length characteristic of PICCs as well as its capability to mechanically inhibit formation of occlusions as set forth herein.

Notwithstanding the foregoing, it should be understood that the plurality of piezoelectric transducers 112 can be alternatively collectively addressed by way of shared electrical leads 114. Collectively addressing the plurality of piezoelectric transducers 112 is simpler for design and manufacture of the catheter 100, but collectively addressing the plurality of piezoelectric transducers 112 foregoes the benefit of being able to customize the adhesion-preventing vibrations along the length of the catheter tube 108 with respect to frequency, amplitude, or pulse waveform by location or specific sites of the individual piezoelectric transducers or the individual groups of piezoelectric transducers. Further, collectively addressing the plurality of piezoelectric transducers 112 can, in some embodiments, forego the benefit of being able to cut the catheter 100 to length while maintaining its capability to mechanically inhibit formation of occlusions as set forth herein.

By way of the piezoelectric effect, the plurality of piezoelectric transducers 112 can be further configured as a plurality of sensors for sensing and, thereby, monitoring environmental conditions of the catheter tube 108 when the catheter tube 108 is placed in the vasculature. Such environmental conditions can include fluid pressure, fluid flowrate, temperature, infiltration or extravasation in or around the catheter 100, heart rate, acoustic signals from the heart (e.g., heart murmurs), or the like.

As shown in FIG. 3, the controller 104 and the internal power source 106 can be incorporated into a portion of the catheter hub 102. Optionally, the controller 104 and the internal power source 106 can be incorporated into a suture wing 116 of the catheter hub 102. Regardless, as shown in FIG. 7, the controller 104 can include components selected from at least a processor 118, secondary memory 120, and a sensor interface 122.

The processor 118 can include a control unit 124, an arithmetic unit 126, and primary memory 128 (e.g., cache memory, random-access memory [“RAM”], or both), wherein the primary memory 128 can be configured to store in-use programs and data (e.g., the sensor data). While the primary memory 128 can be within a same package as a remainder of the processor 118 as alluded to in FIG. 7, at least the foregoing RAM can be distributed outside the package of the processor 118, for example, in its own package. When the plurality of piezoelectric transducer are configured in whole or in part as the plurality of sensors, the processor 118 can further include an analog-to-digital converter (“ADC”) 130 configured to convert electrical signals from the plurality of piezoelectric sensors from analog to digital and a digital-signal processor (“DSP”) 132 configured to generate sensor data from the electrical signals. While the ADC 130 and DSP 132 and can be within a same package as a remainder of the processor 118 as alluded to in FIG. 7, the ADC 130 and DSP 132 and can be distributed outside the package of the processor 118, for example, in their own package.

The secondary memory 120 can be configured to store data (e.g., the sensor data) and programs including instructions, logic including vibrating or sensing logic, algorithms including vibrating or sensing algorithms, or some combination thereof for loading into the primary memory 128 for use by the processor 118, for example, when modulating driving voltages for customizing the adhesion-preventing vibrations or determining from the sensor data, the environmental conditions of the catheter tube 108 when the catheter tube 108 is placed in the vasculature. Indeed, excitation of the plurality of piezoelectric transducers 112 can be in accordance with any mode or combination of modes selected from continuous mode, pulsed mode, or burst mode, which modes for excitation of the plurality of piezoelectric transducers 112 are stored in the secondary memory 120 for loading into the primary memory 128 for use by the processor 118, as needed, for preventing adhesion of thrombotic or non-thrombotic occlusions or eliminating any existing buildup.

The sensor interface 122 can include a signal conditioner 134 configured to standardize the electrical signals from the plurality of piezoelectric sensors through voltage or current limiting, anti-aliasing filtering, or the like. In addition, the sensor interface 122 can include an amplifier 136 configured to amplify the electrical signals and, thereby, increase their signal-to-noise ratio.

The internal power source 106 can be configured to power the controller 104 and the plurality of piezoelectric transducers 112. Such an internal power source 106 can be an internal battery, which internal battery can be rechargeable by way of a port in the catheter hub 102. Optionally, the internal battery is a lithium-ion battery, coin-cell battery, or a flexible battery for use with a remainder of the flexible electronics. Notwithstanding the foregoing, the internal power source 106 can alternatively be an external power source configured for wired or wireless power.

The controller 104 can be configured to control at least the plurality of piezoelectric transducers 112, whether the plurality of piezoelectric transducers 112 are configured as the plurality of vibrators, the plurality of sensors, or both the plurality of vibrators and the plurality of sensors. When the plurality of piezoelectric transducers 112 are configured as both the plurality of vibrators and the plurality of sensors a portion of the plurality of piezoelectric transducers 112 can be exclusively configured as the plurality of vibrators and another portion of the plurality of piezoelectric transducers 112 can be exclusively configured as the plurality of sensors. Alternatively, the plurality of piezoelectric transducers 112 can be configured to independently operate as vibrators or sensors depending on control signals provided thereto by the controller 104.

When the plurality of piezoelectric transducers 112 are configured in whole or in part as the plurality of vibrators, the controller 104 can be configured to vibrate the plurality of vibrators at a frequency of at least about 30 Hz, including at least about 1 kHz, such as at least about 20 kHz, for example, at least about 50 kHz, 100 kHz, 150 kHz, 200 kHz, 250 kHz, 500 kHz, 5 MHz, or 15 MHz. Alternatively, the controller 104 can be configured to vibrate the plurality of vibrators at a frequency of no more than about 15 MHz, including no more than about 5 MHz, such as no more than about 500 kHz, for example, no more than about 250 kHz, 200 kHz, 150 kHz, 100 kHz, 50 kHz, 20 kHz, 1 kHz, or 30 Hz. For example, the controller 104 can be configured to vibrate the plurality of vibrators in a frequency range from about 30 Hz to about 15 MHz, including from about 1 kHz to about 5 MHz, such as from about 20 kHz to about 250 kHz, for example, from about 20 kHz to about 150 kHz.

Further when the plurality of piezoelectric transducers 112 are configured in whole or in part as the plurality of vibrators, the controller 104 can be configured to vibrate the plurality of vibrators with an amplitude of at least about 1 nm, including at least about 100 nm, such as at least about 200 nm, for example, at least about 300 nm, 400 nm, 500 nm, 10 μm, or 100 μm. Alternatively, the controller 104 can be configured to vibrate the plurality of vibrators with an amplitude of no more than about 100 μm, including no more than about 10 μm, such as no more than about 500 nm, for example, no more than about 400 nm, 300 nm, 200 nm, 100 nm, or 1 nm. For example, the controller 104 can be configured to vibrate the plurality of vibrators with an amplitude range from about 1 nm to about 100 μm. Notably, constructive interference from superposition of vibrational waves of at least adjacent piezoelectric transducers can advantageously increase amplitudes, as desired, in one or more locations or specific sites along the length of the catheter tube 108 to inhibit buildup of thrombotic or non-thrombotic occlusions.

Notably, power consumption of the plurality of piezoelectric transducers 112, which depends on a combination of dimensions, vibrational frequencies, and vibrational amplitudes of the piezoelectric transducers can range from about 1 μW to about 100 mW.

### Catheter Assemblies

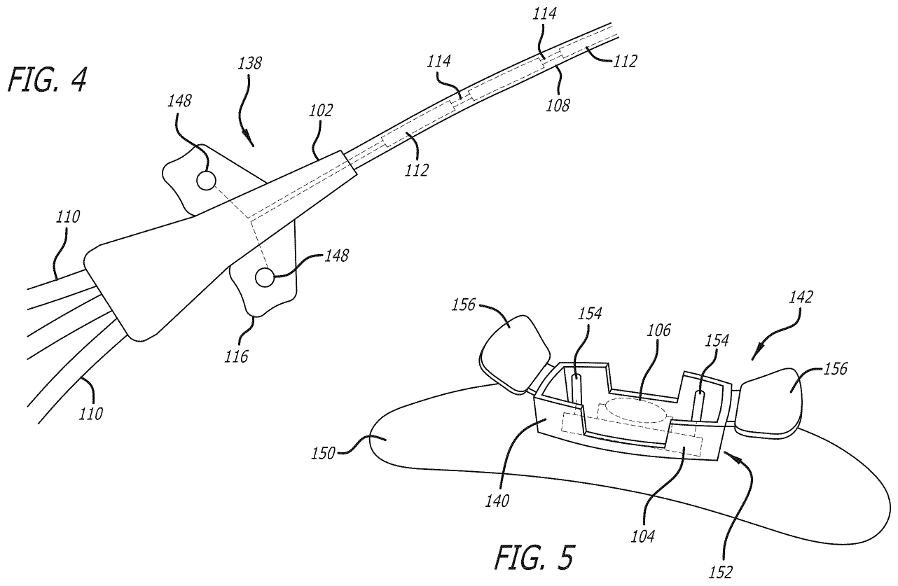

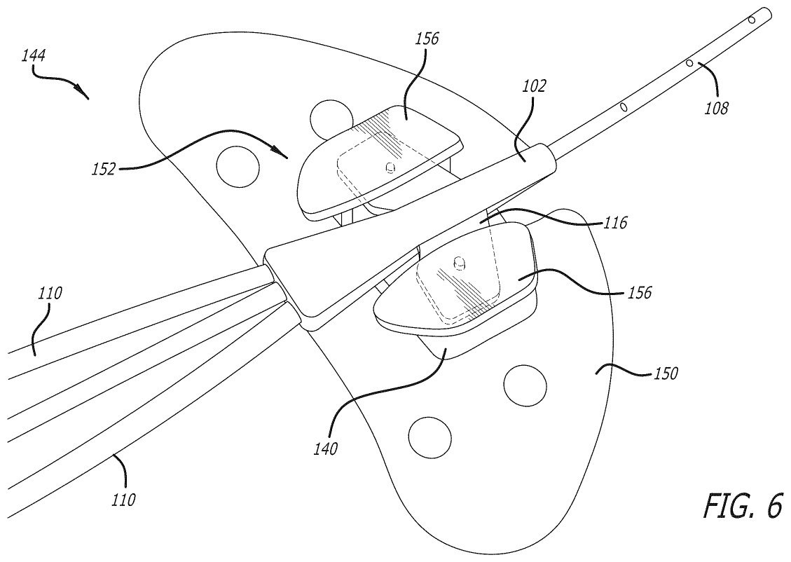

FIG. 4 illustrates another catheter 138 for mechanically inhibiting formation of occlusions in accordance with some embodiments, and FIG. 5 illustrates a detailed view of a base plate 140 of a catheter securement device 142 for the catheter 138, the catheter securement device 142 including the controller 104 and the internal power source 106 incorporated into the base plate 140 in accordance with some embodiments. FIG. 6 illustrates the catheter 138 secured in the catheter securement device 142, thereby forming a catheter assembly 144 for mechanically inhibiting formation of occlusions in accordance with some embodiments. FIG. 7 illustrates a block diagram of the catheter assembly 144 of FIG. 6 in accordance with some embodiments.

As shown in FIGS. 4-6, the catheter assembly 144 can include the catheter 138 and the catheter securement device 142, wherein the plurality of piezoelectric transducers 112, the controller 104, and the internal power source 106 are distributed between separate components of the catheter assembly 144, namely the catheter 138 and the catheter securement device 142.

Like the catheter 100 set forth above, the catheter 138 of FIGS. 4 and 6 can include the catheter tube 108, the catheter hub 102, and the one-or-more extension legs 110 coupled together in the foregoing order. Further as set forth above, the catheter tube 108 can include the plurality of piezoelectric transducers 112 as well as the electrical leads 114 along the length of the catheter tube 108. Different than the catheter 100 set forth above, the catheter 138 need not include the controller 104 or the internal power source 106, which are provided in the catheter securement device 142 as set forth below. Accordingly, the catheter 138 can be configured such that suture-wing through holes 146 of the suture wing 116 include ring contacts 148 operably connected to the plurality of piezoelectric transducers 112 by the electrical leads 114 for powering and controlling the plurality of piezoelectric transducers 112 of the catheter tube 108.

As shown in FIG. 5, the catheter securement device 142 can include a pad 150 and a retainer 152 fixedly coupled together, the catheter securement device 142 configured for securing the catheter 138 to the patient while the catheter tube 108 of the catheter 138 is placed within the vasculature of the patient.

The pad 150 can include a skin-facing side including an adhesive thereon for adhering the catheter securement device 142 to the patient after a liner over the skin-facing side of the pad 150 is removed. The pad 150 can also include an exposed side opposite the skin-facing side of the pad 150 to which the retainer 152 is fixedly attached.

The retainer 152 can include the base plate 140, a pair of posts 154 extending from the base plate 140, and a top cover 156, wherein the base plate 140, the top cover 156, or both the base plate 140 and the top cover 156 include the controller 104 and the internal power source 106. Indeed, as shown in FIG. 5, both the controller 104 and the internal power source 106 can be incorporated into the base plate 140 of the retainer 152; however, the controller 104 and the internal power source 106 can be alternatively distributed between the base plate 140 and the top cover 156 in some embodiments. Whether the controller 104 and the internal power source 106 are incorporated into the base plate 140, the top cover 156, or distributed between the base plate 140 and the top cover 156 of the catheter securement device 142, the catheter securement device 142 can include securement device-based electrical connectors in the base plate 140 or top cover 156 for connecting to catheter-based electrical connectors for powering and controlling the plurality of piezoelectric transducers 112. For example, the pair of posts 154 extending from the base plate 140 can be configured as the catheter-based electrical connectors for connecting to the ring contacts 148 of the suture wing 116 of the catheter 138 as the catheter-based electrical connectors for powering and controlling the plurality of piezoelectric transducers 112 of the catheter tube 108 of the catheter 138. The top cover 156, which is shown in FIGS. 5 and 6 as formed of two living-hinged top-cover pieces, can be configured to cover the suture wing 116 of the catheter 138 and hold it in place over the pair of posts 154 extending from the base plate 140, thereby maintaining sufficient electrical contact for powering and controlling the plurality of piezoelectric transducers 112.

Notably, while the catheter assembly 144 can include the plurality of piezoelectric transducers 112, the controller 104, and the internal power source 106 distributed between separate components of the catheter assembly 144, namely the catheter 138 and the catheter securement device 142, the catheter assembly 144 need not be limited to the foregoing configuration. Indeed, the controller 104 and the internal power source 106 can instead be housed in an independent controller unit unrelated to the catheter securement device 142. Such a controller unit can be configured with a plug or the like to plug into a port of the catheter hub 102 of the catheter 138, which controller unit can be easily swapped for another, charged controller unit, as needed, to maintain mechanical inhibition over formation of occlusions in the catheter tube 108 of the catheter 138 with minimal interruption. That said, the plurality of piezoelectric transducers 112, the controller 104, and the internal power source 106 can be incorporated into a single-component catheter assembly such as the catheter 100 set forth above.

### Methods

Methods can include methods of using and making the catheter 100 or catheter assembly 144.

As to a method of using the catheter 100 or the catheter assembly 144, such a method can include one or more operations selected from an excising operation, a placing operation, a securing operation, a powering operation, and a customizing operation.

The excising operation can be before the placing operation, as the excising operation can include excising a distal-end portion of the catheter tube 108 for a desired placement length of the catheter tube 108. As set forth above, the individual piezoelectric transducers, the individual groups of piezoelectric transducers, or the combination of the individual piezoelectric transducers and the individual groups of piezoelectric transducers being individually electronically addressed allows remaining piezoelectric transducers of the plurality of piezoelectric transducers 112 not excised with the distal-end portion of the catheter tube 108 to vibrate along the placement length of the catheter tube 108 in accordance with being individually electronically addressed.

The placing operation can include placing the catheter 100 or 138 in a vasculature of a patient such as by the Seldinger technique.

The securing operation can include securing the catheter 100 or 138 to the patient. With respect to the catheter 138, the securing operation can include securing the catheter 138 in the catheter securement device 142, which, in turn, can include disposing the suture wing 116 of the catheter 138 in the base plate 140 over the pair or posts 154 extending therefrom such that the ring contacts 148 of the suture wing 116 make sufficient electrical contact for powering and controlling the plurality of piezoelectric transducers 112. In addition, the securing operation can include covering the suture wing 116 of the catheter 138 to hold it in place over the pair of posts 154 and maintain the electrical contact for powering and controlling the plurality of piezoelectric transducers 112.

The powering operation can include powering up the catheter 100 or the catheter assembly 144 by way of the internal power source 106. Such a powering operation activates the controller 104 including the processor 118 and memory to control the plurality of piezoelectric transducers 112, thereby vibrating and, thusly, inhibiting buildup of biomaterial on the luminal surface of the catheter tube 108, the abluminal surface of the catheter tube 108, or both the luminal and abluminal surfaces of the catheter tube 108 by way of the adhesion-preventing vibrations along the length of the catheter tube 108.

The customizing operation can include imaging the catheter 100 or 138 via ultrasound or the like, determining where a thrombotic or non-thrombotic occlusion is located in the catheter tube 108, if such an occlusion is present, and applying customized vibrations where the thrombotic or non-thrombotic occlusion is located in the catheter tube 108. The customized vibrations can be customized with respect to frequency, amplitude, or pulse waveform, and the custom vibrations can be provided where the thrombotic or non-thrombotic occlusion is located in the catheter tube 108 by one or more individual piezoelectric transducers or one or more individual groups of piezoelectric transducers. Further, the burst mode set forth above can be used in the customized vibrations to provide vibrational frequencies up to the MHz range and vibrational amplitudes up to the μm range in order to break up the thrombotic or non-thrombotic occlusion located in the catheter tube 108. Advantageously, like any adhesion-preventing vibrations provided along the length of the catheter tube 108 set forth herein, such customized vibrations can be confined to the luminal or abluminal surface of the catheter tube 108 as surface waves thereon, thereby selectively targeting either the luminal or abluminal surface of catheter tube 108 over the other.

Advantageously, once the catheter 100 or 138 is placed and the catheter 100 or the catheter assembly 144 is powered up in accordance with the placing and powering operations, respectively, no further intervention (e.g., catheter flushes, antibiotic lock therapies, etc.) by a clinician is necessary until the internal power source 106 is recharged or replaced. Further, being that the internal power source 106 obviates the patient being connected to an external power source, the patient can enjoy as much mobility as the patient is allowed to enjoy.

As to a method of making the catheter 100 or the catheter assembly 144, such a method can include one or more operations selected from a fabricating operation and an assembling operation.

The fabricating operation can include fabricating the flexible electronics on the luminal surface of the catheter tube 108, fabricating the flexible electronics on the abluminal surface of the catheter tube 108, or disposing the flexible electronics between the luminal and abluminal surfaces of the catheter tube 108. Such fabricating of the flexible electronics can include using a contact-type flexible electronics fabrication method selected from screen printing, flexographic printing, gravure printing, and soft lithographic printing. Alternatively, the fabricating of the flexible electronics can include using a non-contact-type flexible electronics fabrication method selected from multiphoton lithography or direct laser-writing lithography, aerosol printing, and inkjet printing. In accordance with that set forth above for the flexible electronics fabricated on the luminal surface of the catheter tube 108, the flexible electronics can be fabricated on the tubular substrate or even as the tubular substrate, and a remainder of the catheter tube 108 can be extruded or dip coated thereover. And in accordance with that set forth above for the flexible electronics disposed between the luminal and abluminal surfaces of the catheter tube 108, the flexible electronics can be fabricated on an inner layer of the catheter tube 108, and an outer layer of the catheter tube 108 can be extruded or dip coated thereover.

The assembling operation can include inserting or otherwise disposing the proximal-end portion of the catheter tube 108 into the catheter hub 102. Further, the assembling operation can include inserting or otherwise disposing the distal-end portion of each extension leg of the one-or-more extension legs 110 into the catheter hub 102.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A catheter assembly, comprising:
a catheter including:
a catheter tube incorporating a plurality of piezoelectric transducers into a length of the catheter tube, the plurality of piezoelectric transducers configured as a plurality of vibrators for vibrating and, thereby, inhibiting buildup of biomaterial on a luminal surface of the catheter tube, an abluminal surface of the catheter tube, or both the luminal and abluminal surfaces of the catheter tube by way of vibrations along the length of the catheter tube when the catheter tube is placed in a vasculature; and
a catheter hub, a proximal-end portion of the catheter tube disposed in the catheter hub;
a controller including a processor and memory, the controller configured to control at least the plurality of piezoelectric transducers; and
an internal power source configured to power the controller and the plurality of piezoelectric transducers.

2. The catheter assembly of claim 1, wherein the plurality of piezoelectric transducers and electrical leads connecting the plurality of piezoelectric transducers to the controller are flexible electronics fabricated on the luminal surface of the catheter tube, fabricated on the abluminal surface of the catheter tube, or disposed between the luminal and abluminal surfaces of the catheter tube.

3. The catheter assembly of claim 2, wherein the internal power source is a lithium-ion battery or a flexible battery.

4. The catheter assembly of claim 2, wherein a thickness of the flexible electronics ranges from about 1 μm to about 500 μm.

5. The catheter assembly of claim 1, wherein individual piezoelectric transducers of the plurality of piezoelectric transducers, individual groups of piezoelectric transducers of the plurality of piezoelectric transducers, or a combination of the individual piezoelectric transducers and the individual groups of piezoelectric transducers are individually electronically addressed.

6. The catheter assembly of claim 5, wherein the individual piezoelectric transducers, the individual groups of piezoelectric transducers, or the combination of the individual piezoelectric transducers and the individual groups of piezoelectric transducers being individually electronically addressed allows the vibrations along the length of the catheter tube to be customized by location of the individual piezoelectric transducers or the individual groups of piezoelectric transducers.

7. The catheter assembly of claim 5, wherein the individual piezoelectric transducers, the individual groups of piezoelectric transducers, or the combination of the individual piezoelectric transducers and the individual groups of piezoelectric transducers being individually electronically addressed allows a distal-end portion of the catheter tube to be excised for a desired placement length of the catheter tube, remaining piezoelectric transducers of the plurality of piezoelectric transducers capable of vibrations along the placement length of the catheter tube in accordance with being individually electronically addressed.

8. The catheter assembly of claim 1, wherein the plurality of piezoelectric transducers are further configured as a plurality of sensors for sensing and, thereby, monitoring environmental conditions of the catheter tube when the catheter tube is placed in the vasculature.

9. The catheter assembly of claim 1, wherein the controller and the internal power source are incorporated into a portion of the catheter hub, optionally, a suture wing of the catheter hub.

10. The catheter assembly of claim 1, wherein the controller and the internal power source are incorporated into a base plate or top cover of a catheter securement device, the base plate or top cover of the catheter securement device including securement device-based electrical connectors for connecting to catheter-based electrical connectors for powering and controlling the plurality of piezoelectric transducers.

11. The catheter assembly of claim 1, wherein the controller is configured to vibrate the plurality of piezoelectric transducers in a frequency range from about 30 Hz to about 15 MHz with an amplitude range from about 1 nm to about 100 μm.

12. The catheter assembly of claim 1, wherein the buildup of biomaterial is an intraluminal thrombus, a fibrin tail, a fibrin sheath, a mural thrombus, a biofilm, or a combination thereof.

13. The catheter assembly of claim 1, wherein the catheter is a central venous catheter (“CVC”) or a peripherally inserted central catheter (“PICC”).

14. A method of catheter assembly, comprising:
placing a catheter of the catheter assembly in a vasculature of a patient, the catheter including:
a catheter tube incorporating a plurality of piezoelectric transducers into a length of the catheter tube, the plurality of piezoelectric transducers configured as a plurality of vibrators; and
a catheter hub, a proximal-end portion of the catheter tube disposed in the catheter hub; and
powering up the catheter assembly by way of an internal power source, the powering up of the catheter assembly activating a controller including a processor and memory configured to control the plurality of piezoelectric transducers, thereby vibrating and, thusly, inhibiting buildup of biomaterial on a luminal surface of the catheter tube, an abluminal surface of the catheter tube, or both the luminal and abluminal surfaces of the catheter tube by way of vibrations along the length of the catheter tube.

15. The method of claim 14, wherein the plurality of piezoelectric transducers and electrical leads connecting the plurality of piezoelectric transducers to the controller are flexible electronics fabricated on the luminal surface of the catheter tube, fabricated on the abluminal surface of the catheter tube, or disposed between the luminal and abluminal surfaces of the catheter tube.

16. The method of claim 14, wherein individual piezoelectric transducers of the plurality of piezoelectric transducers, individual groups of piezoelectric transducers of the plurality of piezoelectric transducers, or a combination of the individual piezoelectric transducers and the individual groups of piezoelectric transducers are individually electronically addressed.

17. The method of claim 16, wherein the individual piezoelectric transducers, the individual groups of piezoelectric transducers, or the combination of the individual piezoelectric transducers and the individual groups of piezoelectric transducers being individually electronically addressed allows the vibrations along the length of the catheter tube to be customized by location of the individual piezoelectric transducers or the individual groups of piezoelectric transducers.

18. The method of claim 16, further comprising excising a distal-end portion of the catheter tube for a desired placement length of the catheter tube before the placing of the catheter in the vasculature of a patient, the individual piezoelectric transducers, the individual groups of piezoelectric transducers, or the combination of the individual piezoelectric transducers and the individual groups of piezoelectric transducers being individually electronically addressed allowing remaining piezoelectric transducers of the plurality of piezoelectric transducers to vibrate along the placement length of the catheter tube after the powering up the catheter assembly in accordance with being individually electronically addressed.

19. The method of claim 14, wherein the plurality of piezoelectric transducers are further configured as a plurality of sensors for sensing and, thereby, monitoring environmental conditions of the catheter tube upon the placing of the catheter in the vasculature of the patient, the powering up of the catheter assembly, and the activating of the controller.

20. The method of claim 14, wherein the controller and the internal power source are incorporated into a portion of the catheter hub, optionally, a suture wing of the catheter hub.

21. The method of claim 14, wherein the buildup of biomaterial is an intraluminal thrombus, a fibrin tail, a fibrin sheath, a mural thrombus, a biofilm, or a combination thereof.
